(19) United States
(12) Patent Application Publication
(10) Pub. No.: US 2005/0004553 A1
(43) Pub. Date: Jan. 6, 2005

(54) Sheath catheter having variable over-the-wire length and methods of use

(75) Inventor: Nareak Douk, Lowell, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic AVE, Inc.

(21) Appl. No.: 10/610,817

(22) Filed: Jul. 2, 2003

(51) Int. Cl. A61M 25/00

(52) U.S. Cl. 604/523

(57) ABSTRACT

A sheath catheter includes a positioning wire fixedly attached at its distal end to a distal tube. A proximal tube is slidably disposed on the positioning wire proximal of the distal tube and sized so that the distal tube may be nested therein. In one embodiment, a series of coordinating stops are fixed on the tubes so that the distal tube can slide back and forth within the proximal tube but cannot be extracted from the proximal tube. As such, the over-the-wire length of the catheter can be varied. In another embodiment, an additional telescoping portion can be added between the distal tube and the proximal tube for greater control over the length of the sheath catheter. In this embodiment, the tubes are sized so that the middle tube may be nested within the proximal tube and the distal tube may be nested in the middle tube.

### BACKGROUND OF THE INVENTION

1. Field of the Invention

This invention relates to catheters for use within a body of a patient, and more particularly to sheath catheters for use in deploying and retrieving therapeutic or interventional medical devices.

2. Background of the Invention

Catheters have long been used for the treatment of diseases of the cardiovascular system, such as treatment or removal of stenosis. For example, in a percutaneous transluminal coronary angioplasty (PTCA) procedure, a catheter is used to insert a balloon into a patient's cardiovascular system, position the balloon at a desired treatment location, inflate the balloon, and remove the balloon from the patient. Another example is the placement of a prosthetic stent that is placed in the body on a permanent or semi-permanent basis to support weakened or diseased vascular walls to avoid catastrophic closure or rupture thereof.

Often, more than one interventional catheter is used during a procedure, such as to change the size of the balloon being used or to introduce additional devices into the system to aid with the procedure. In such situations, the catheters are generally inserted into the patient's cardiovascular system with the assistance of a guidewire. In one instance, a guidewire is introduced into the patient, steered through the tortuous pathways of the cardiovascular system, and positioned at a predetermined location. Various catheters having a guidewire lumen adapted to receive the guidewire may then be introduced into and removed from the patient along the guidewire, thereby decreasing the time needed to complete a procedure.

Alternatively, the guidewire may be introduced into the system with a deployment catheter already in place. Deployment catheters are also termed “sheath” catheters in the art. This is often the case when the guidewire includes a device along its length, such as a self-expanding distal protection filter placed downstream of the treatment area for filtering and removing embolic material that may become dislodged during a procedure. The deployment catheter gives the guidewire a low profile, which helps to advance the guidewire through the narrow and tortuous pathway of the cardiovascular system. After the guidewire has been advanced to the target location, the sheath catheter is removed, which allows the filter to expand within the body lumen, and other therapeutic catheters are then introduced over the guidewire.

A sheath catheter can also be used as a retrieval catheter at the end of a procedure to reduce the profile of a filter and to ease removal of the filter. Referring to the example noted above with respect to a self-expanding filter, the filter is somewhat expanded due to the embolic particles collected therein. A retrieval catheter may be used to close the filter and smooth the profile thereof so that the guidewire may pass through the treatment area without disturbing any stents or otherwise interfering with the treated vessel.

These catheters are of the “over-the-wire” variety, with a guidewire lumen extending the entire length of the catheter. The guidewire is disposed entirely within the catheter except for the distal and proximal portions of the guidewire which protrude from the catheter. While these catheters are advantageous in many ways, exchanging the indwelling catheter for another interventional or the retrieval catheter can be difficult. In order to maintain a guidewire in position while withdrawing the indwelling catheter, the clinician must grip the proximal end of the guidewire to prevent it from becoming dislodged during removal of the indwelling catheter. However, the catheter, which is typically on the order of 135 centimeters long, is generally longer than the exposed portion of the guidewire. Therefore, to be able to maintain the guidewire in place, the guidewire must be sufficiently long so that the clinician may be able to grip an exposed portion of the guidewire. For catheters on the order of 135 centimeters in length, therefore, a guidewire of 300 centimeters in length is necessary. Manipulating a catheter along such a long guidewire typically requires more than one operator, thereby increasing the time and complexity of the procedure.

Many techniques have been used to overcome this problem. For example, a guidewire of a shorter length is used during the procedure, but during the exchange process, a longer exchange guidewire is substituted for the original guidewire. Also, as is disclosed in U.S. Pat. No. 4,917,103 to Gambale et al., incorporated herein in its entirety by reference thereto, the length of the original guidewire may be extended using a guidewire extension apparatus. However, neither of these techniques eliminate the need for more than one operator to complete the procedure.

Various techniques have also focused on adjusting the length of the catheter, so that the length thereof can be reduced when necessary. U.S. Pat. No. 5,591,194 to Berthiaume (“Berthiaume”), incorporated herein in its entirety by reference thereto, describes an over-the-wire balloon catheter with an adjustable length. The balloon catheter includes several telescoping portions slidably mounted on an inflation shaft which is fixedly attached to the distal balloon. The telescoping portions may be retracted by drawing the inflation shaft proximally, thereby reducing the effective over-the-wire length of the telescoping balloon catheter. As such, the balloon catheter may be withdrawn from the patient without using an unnecessarily long guidewire. However, this patent does not disclose adapting the telescoping catheter technology for use in a deployment or retrieval sheath catheter. In particular, the balloon catheter in Berthiaume uses a multiple lumen design, where at least one lumen is capable of being used as an inflation lumen. Further, the portion on which the balloon is mounted utilizes a complicated structure, so that the balloon may be inflated while still having a guidewire lumen.

### SUMMARY OF THE INVENTION

Accordingly, disclosed herein is a sheath catheter for use as a deployment and/or retrieval catheter. The sheath catheter includes “telescoping” proximal and distal tubes with a positioning wire fixedly attached at its distal end to the distal tube. Each tube has a single lumen, where the proximal tube lumen has an inner diameter that is larger than an outer diameter of the distal tube. The proximal tube is slidably disposed over the positioning wire proximal of the distal tube. The distal tube is sized to slide proximally and distally within the proximal tube lumen when the positioning wire is manipulated. Further, the distal tube cannot be completely extracted from the proximal tube lumen. As such, the catheter can be placed in an expanded position either by pulling the distal tube or by pushing the positioning wire thereby causing the distal tube to be moved distally. The catheter can be retracted to a rapid exchange length by pulling the positioning wire proximally so that the distal tube is drawn into the proximal tube lumen thereby shortening the effective over-the-wire length of the catheter.

### BRIEF DESCRIPTION OF THE DRAWINGS/FIGURES

The accompanying drawings, which are incorporated herein and form a part of the specification, illustrate the present invention and, together with the description, further serve to explain the principles of the invention and to enable a person skilled in the pertinent art to make and use the invention.

FIG. 1 is a longitudinal cross-sectional view of a telescoping sheath catheter according to the present invention in a fully extended position.

FIG. 2 is an enlarged view of the joint area of a proximal tube and a distal tube of the catheter of FIG. 1.

FIG. 3 is a schematic view of the catheter of FIG. 1 in the rapid exchange position.

FIG. 4 is a longitudinal cross-sectional view of an alternate embodiment of a telescoping sheath catheter according to the present invention in a fully extended position.

FIG. 5 is a longitudinal cross-sectional view of the catheter of FIG. 4 in the rapid exchange position.

### DETAILED DESCRIPTION OF THE INVENTION

Specific embodiments of the present invention are now described with reference to the figures, where like reference numbers indicate identical or functionally similar elements.

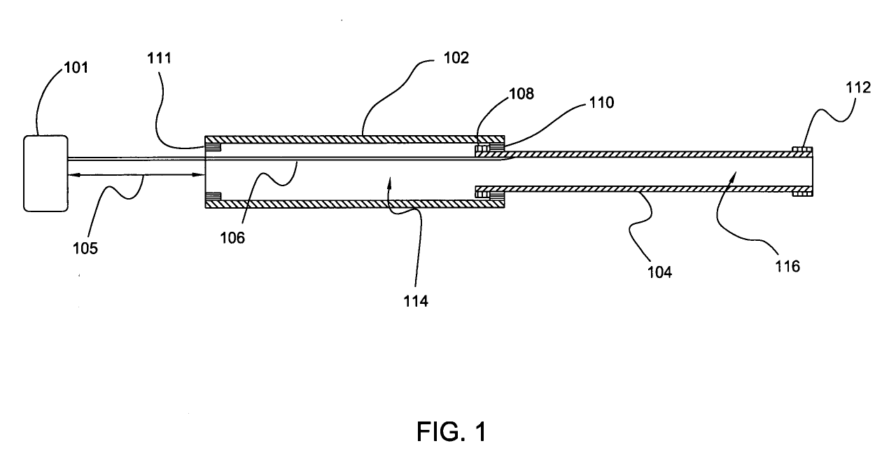

Referring now to FIG. 1, a telescoping sheath catheter 100 is shown. Catheter 100 includes a grip portion 101, a positioning wire 106, a proximal tubular element 102, and a distal tubular element 104. Proximal tubular element 102 is open at both ends with a lumen 114 extending therethrough. Distal tubular element 104 is also open at both ends with a lumen 116 extending therethrough.

Proximal tubular element 102 and distal tubular element 104 are preferably made of polymeric materials suitable for placement in a patient's body, such as polyvinyl chloride, polyethylene, polyethylene terephthalate, polyamide, or, preferably, polyimide. Further, an optional layer of a stiffer material may be added to or embedded within the main material of proximal tubular element 102 and distal tubular element 104 to enhance the pushability of catheter 100. For example, a braid of metal or polymeric filaments could be included. Proximal tubular element 102 and distal tubular element 104 can be manufactured by any method known in the art, such as by extrusion.

Additionally, distal tubular element 104 optionally includes at a distal end thereof a radiopaque band 112 to allow for easy tracking of the progress of catheter 100 through a patient's system. In one embodiment, radiopaque band 112 is a short length of platinum tubing affixed to the distal end of distal tubular element 104, such as by cementing or heat bonding.

The diameter of lumen 116 of distal tubular element 104 is sized so as to fit over a collapsed device mounted on a therapeutic catheter, such as a collapsed distal protection filter or any other type of self-expanding distal protection element, such as an occluder. As shown in FIG. 1, the diameter of lumen 114 of proximal tubular element 102 is greater than an outer diameter of distal tubular element 104. As such, distal tubular element 104 may be slidably received within proximal tubular element 102. The dimensions in FIG. 1 are exaggerated for clarity; in actual use, the inner diameter of proximal tubular element 102 and the outer diameter of distal tubular element 104 differ by a fairly small degree. Further, proximal tubular element 102 and distal tubular element 104 have relatively thin walls, so as to minimize the discontinuity at the joint on an exterior surface of catheter 100.

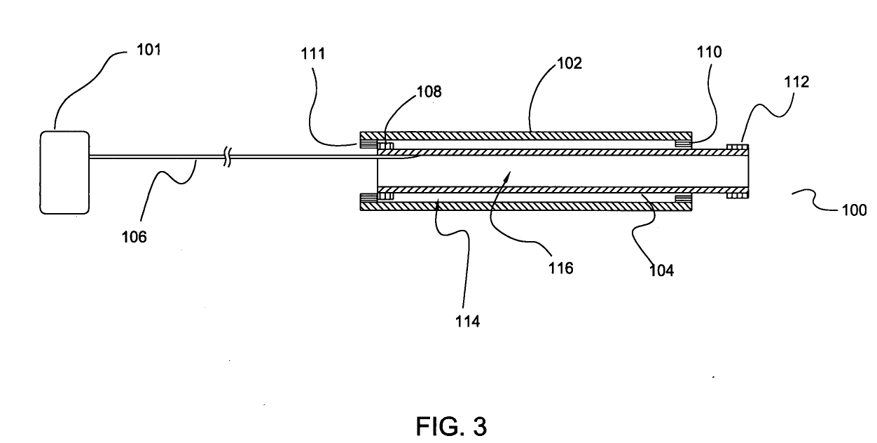

The lengths of proximal tubular element 102 and distal tubular element 104 are approximately equal. While the actual lengths thereof may vary widely, the total length of catheter 100 when fully contracted (as seen in FIG. 3) is substantially less than that of the typical guidewire. For the purposes of illustration only, a typical PTCA or coronary interventional catheter is approximately 135 cm long. In this case, proximal tubular element 102 and distal tubular element 104 would each be approximately 70 cm in length, to compensate for the overlap between the two portions.

As shown in FIG. 1, positioning wire 106 extends from grip portion 101, through lumen 114 of proximal tubular element 102, into lumen 116 of distal tubular element 104. Positioning wire 106 is a long, thin wire, such as a guidewire or a core wire. As catheter 100 may be extended from a fully nested condition to a fully extended condition in vivo, positioning wire 106 must be sufficiently stiff as to push distal tubular element through the tortuous vasculature and yet flexible enough to navigate the same tortuous vasculature. Any material known in the art for use as a guidewire is appropriate for positioning wire 106. Examples of such materials include stainless steel, nitinol alloys, or polymeric materials. In one embodiment, positioning wire 106 is a solid wire. In another embodiment, positioning wire 106 is a hollow tube.

The length of positioning wire 106 may vary depending upon the design, but positioning wire 106 must be of sufficient length such that a proximal portion 105 thereof extends proximally of a proximal end of proximal tubular element 102, even when catheter 100 is fully extended. For the purposes of illustration only, if catheter 100 is 135 cm, the typical length of a PTCA or coronary intervention catheter, then positioning wire 106 for such a catheter would be approximately 140 cm.

Proximal tubular element 102 is slidably mounted over positioning wire 106. A distal end of positioning wire 106 is fixedly attached to distal tubular element 104. In one embodiment, as shown in FIG. 1, the distal end of positioning wire 106 is fixedly attached to an inner surface of distal tubular element 104. Alternately, the distal end of positioning wire 106 may be attached to a proximal tip of distal tubular element 104, or even to a proximal stop 108 disposed on a proximal end of distal tubular element 104. The fixed attachment is achieved by any method known in the art, such as by cementing, soldering, or heat bonding. A proximal end of distal tubular element 104 is inserted into a distal end of proximal tubular element 102.

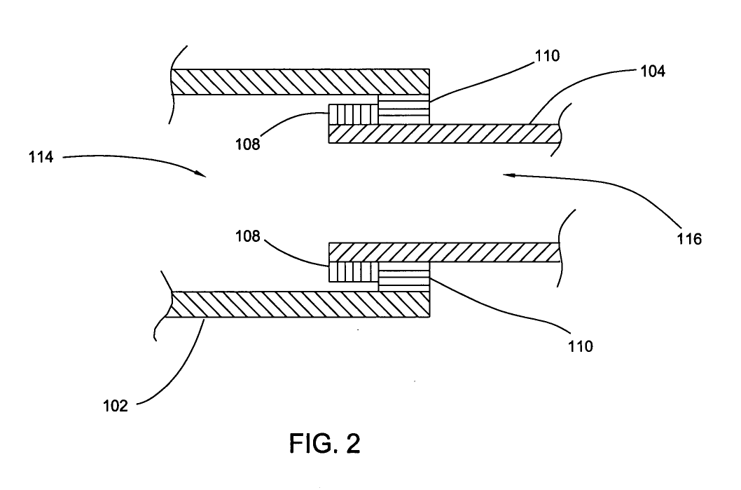

As seen more clearly in FIG. 2, the relative positions of proximal tubular element 102 and distal tubular element 104 are maintained using a series of stops. Proximal stop 108 of distal tubular element 104 is a short length of tubing, which in one embodiment made of the same material as that of distal tubular element 104. Proximal stop 108 is fixedly attached to an outer surface of distal tubular element 104, such as by cementing, soldering, or heat bonding. A distal stop 110 of proximal tubular element 102 is also a short length of tubing, which in one embodiment is made of the same material as that of proximal tubular element 102. Distal stop 110 is bonded to an inner surface of proximal tubular element 102 at the distal end thereof in a similar fashion as proximal stop 108. Proximal stop 108 and distal stop 110 are sized to prevent the removal of the proximal end of distal tubular element 104 from the distal end of proximal tubular element 102. In one embodiment, an outer diameter of proximal stop 108 is approximately equal to the diameter of lumen 114. Similarly, in one embodiment, an inner diameter of distal stop 110 is approximately equal to the outer diameter of distal tubular element 104. As such, proximal stop 108 cannot move past distal stop 110, thereby keeping the proximal end of distal tubular element 104 disposed within proximal tubular element 102. Catheter 100 is in a fully extended or expanded configuration when proximal stop 108 and distal stop 110 abut each other.

Further, as shown in FIG. 3, a stop 111 is positioned to prevent the extraction of distal tubular element 104 from a proximal end of proximal tubular element 102. Stop 111 is also a short length of tubing, preferably made of the same material as that of proximal tubular element 102. Stop 111 is bonded to the inner surface of proximal tubular element 102 at the proximal end thereof, and has a similar inner diameter as distal stop 110.

Although all stops described with respect to FIG. 1 are shown at the proximal or distal ends of proximal tubular element 102 and distal tubular element 104, the placement of the stops need not be so arranged. In order to control the length of catheter 100 in either the fully extended configuration (shown in FIG. 1) or in the nested or contracted configuration (shown in FIG. 3), the stops may be placed anywhere along the lengths of tubular elements 102, 104; however, the placement of the stops on the ends thereof achieves a maximum length for catheter 100. Further, the function of the stops described herein is to prevent the complete extraction of distal tubular element 104 from proximal tubular element 102. However, other structures may be used for this purpose, such as increasing the outer diameter of distal tubular element 104 at the proximal and distal ends thereof, coating the inner surface of proximal tubular element 102 and/or the outer surface of distal tubular element 104 at the proximal and distal ends thereof with a rough material.

Grip portion 101 is a handle for the clinician to grasp and manipulate positioning wire 106. Any such handle known in the art may be used, such as a molded handle with a textured surface for maintaining the grip, or a more ergonomically designed handle with finger holes. Grip portion 101 may be made from any material known in the art for handles, such as plastic, rubber, or metal. Alternatively, grip portion 101 may be eliminated entirely, and the clinician will simply grasp positioning wire 106 directly.

Operation and use of telescoping sheath catheter 100 as a deployment catheter is now described. The specific details included in the following example are for illustrative purposes only; this type of catheter may be used in a similar manner albeit for a slightly different procedure such as introducing diagnostic or therapeutic medical devices, with or without such devices being attached to guidewires. During the course of a typical procedure using a distal protection element, such as a collapsible filter or an occluder, the protection element must reside on the end of a guidewire relative to the treatment position. In order to reduce the profile of the collapsed filter during launch, the filter and guidewire (not shown) are inserted into catheter 100, which is in the fully extended position. The proximal portion of the guidewire extends beyond the proximal end of catheter 100. Catheter 100 and the guidewire are then advanced through a patient's cardiovascular system until the filter is positioned at an appropriate location relative to the treatment location, such as downstream of the treatment location. The sizes of proximal stop 108 and distal stop 110 help to prevent catheter 100 from premature or unintentional contraction during the insertion process.

At this point, catheter 100 is removed from the patient so that the filter may be deployed and other therapeutic and/or diagnostic catheters may be positioned over the guidewire, such as a balloon catheter. To extract catheter 100 quickly and easily, catheter 100 is contracted into a rapid exchange length, shown in FIG. 3. The clinician grasps grip portion 101 and draws positioning wire 106 proximally, pulling distal tubular element 104 into proximal tubular element 102 in a telescoping manner. Distal tubular element 104 is prevented from being pulled through the open proximal end of proximal tubular element 102 by the engagement of proximal stop 108 with stop 111. After the nesting of distal tubular element 104 within proximal tubular element 102 is complete, the effective over-the-wire length of catheter 100 is such that the clinician may withdraw catheter 100 without losing contact with the proximal end of the guidewire.

If the clinician wishes to use catheter 100 as a retrieval catheter for a medical device, such as a filter guidewire assembly, the clinician would first remove any therapeutic catheters from the guidewire. Catheter 100 is in a “nested” condition, as shown in FIG. 3, and the proximal end of the filter guidewire assembly would be positioned within lumen 116. The clinician pushes positioning wire 106 distally to extend catheter 100 to the fully extended position shown in FIG. 1. As it extends, catheter 100 is guided over the guidewire to the filter, which would either be in a collapsed configuration for removal or would be collapsed by passing catheter 100 over the filter. Catheter 100 and the filter guidewire assembly are then removed from the patient as a unit.

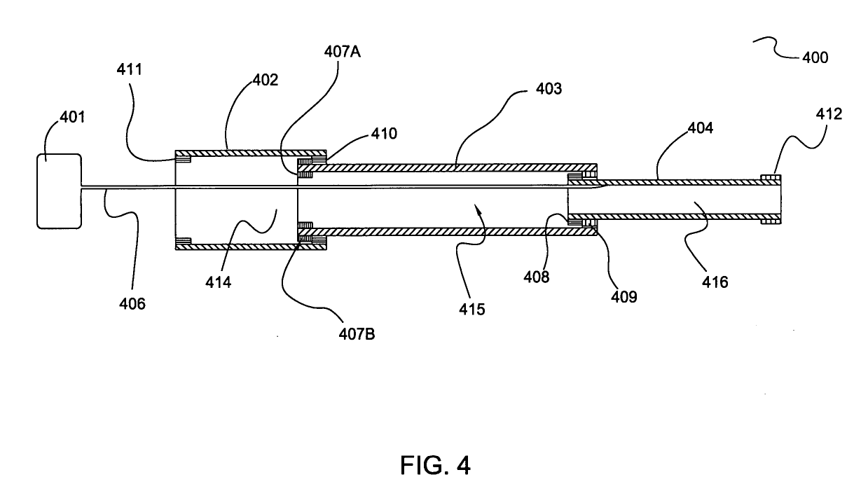

Referring now to FIG. 4, an alternate embodiment of a telescoping sheath catheter 400 is shown. Catheter 400 includes a grip portion 401, a proximal tubular element 402 defining a lumen 414, a middle tubular element 403 defining a lumen 415, and a distal tubular element 404 defining a lumen 416. As seen in FIG. 4, the lumens of the individual tubular elements create, a single, unobstructed lumen that extends the length of catheter 400. In this triple-element embodiment, an effective over-the-wire length of catheter 400 can be reduced to be significantly less than that of the dual-element design of catheter 100. However, if an inner lumen of distal tubular element 404 is the same as that of distal tubular element 104, then an outer diameter of proximal tubular element 402 will be larger than that of proximal tubular element 102 (described above) due to the requisite nesting of both middle tubular element 403 and distal tubular element 404 within proximal tubular element 402.

Catheter 400 is similar in construction to catheter 100. The same or similar materials used to form tubular elements 102, 104 are used to form tubular elements 402, 403, 404. In one embodiment, the material is polyimide. Further, as with catheter 100, in one embodiment, the material used for catheter 400 includes a similar reinforcing layer, such as a metal braid, embedded within the main polymer.

As shown in FIG. 4, a positioning wire 406 extends from grip portion 401, through lumens 414 and 415 to distal tubular element 404, wherein positioning wire 406 is affixed to a proximal end thereof. In one embodiment, shown in FIG. 4, positioning wire 406 extends into lumen 416 and is fixedly attached to an inner wall of distal tubular element 404. Positioning wire 406 is a flexible wire, such as a guidewire or core wire, and any material known in the art for use as a guidewire is appropriate for its use. Examples of such materials include but are not limited to stainless steel or nitinol alloys.

Additionally, distal tubular element 404 optionally includes at a distal end thereof a radiopaque band 412 to allow for easy tracking of the progress of catheter 100 through a patient's system. In one embodiment, radiopaque band 412 is a short length of platinum tubing affixed to the distal end of distal tubular element 404, such as by cementing or heat bonding.

A grip portion 401 is a handle for the clinician to grasp and manipulate positioning wire 406. As with grip portion 101 described above with respect to FIG. 1, any handle known in the art may be used. Alternatively, grip portion 401 may be eliminated entirely, and the clinician will simply grasp positioning wire 406 directly.

As with catheter 100, the relative positions of proximal tubular element 402, middle tubular element 403, and distal tubular element 404 are maintained using a series of stops. As seen in FIG. 4, a proximal stop 408 of distal tubular element 404 is a short length of tubing, which in one embodiment is made of the same material as that of distal tubular element 404. Proximal stop 408 is bonded to an outer surface of distal tubular element 404.

A distal stop 409 of middle tubular element 403 is also a short length of tubing, which in one embodiment is made of the same material as that of middle tubular element 403, bonded to an inner surface of middle tubular element 403. A proximal inner stop 407A of middle tubular element 403 includes a short length of tubing bonded to an inner surface at a proximal end of middle tubular element 403. A proximal outer stop 407B of middle tubular element 403 includes a short length of tubing bonded to an outer surface at the proximal end of middle tubular element 403. In one embodiment, both proximal inner stop 407A and proximal outer stop 407B are made of the same material as middle tubular element 403.

A distal stop 410 of proximal tubular element 402 is also a short length of tubing, which in one embodiment is made of the same material as that of proximal tubular element 402. Distal stop 410 is bonded to an inner surface of proximal tubular element 404 at a distal end thereof.

Proximal outer stop 407B and distal stop 410 are sized to prevent the removal of the proximal end of middle tubular element 403 from the distal end of proximal tubular element 402. In one embodiment, an outer diameter of proximal outer stop 407B is approximately equal to a diameter of lumen 414. Similarly, in one embodiment, an inner diameter of distal stop 410 is approximately equal to an outer diameter of middle tubular element 403.

Proximal outer stop 407B and proximal stop 411 are sized to prevent the removal of the proximal end of middle tubular element 403 from the proximal end of proximal tubular element 402. Thus, in one embodiment, an inner diameter of proximal stop 411 is approximately equal to the outer diameter of middle tubular element 403.

Proximal stop 408 and distal stop 409 are sized to prevent the removal of the proximal end of distal tubular element 404 from the distal end of middle tubular element 403. In one embodiment, an outer diameter of proximal stop 408 is approximately equal to that of lumen 415. Similarly, in one embodiment, an inner diameter of distal stop 409 is approximately equal to an outer diameter of distal tubular element 404.

Proximal inner stop 407A and proximal stop 408 are sized to prevent the removal of the proximal end of distal tubular element 404 from the proximal end of middle tubular element 403.

Catheter 400 is shown in a fully extended or expanded configuration in FIG. 4, when proximal outer stop 407B of middle tubular element 403 and distal stop 410 of proximal tubular element 402 abut one another and proximal stop 408 of distal tubular element 404 and distal stop 409 of middle tubular element 403 abut each other.

Catheter 400 is in a first partially expanded configuration (not shown) when proximal outer stop 407B and distal stop 410 abut one another, but proximal stop 408 and distal stop 409 do not abut. Proximal stop 408 may or may not abut proximal inner stop 407A. In other words, middle tubular element 403 is not disposed within proximal tubular element 402 while distal tubular element 404 is partially or fully nested within middle tubular element 403.

Catheter 400 is in a second partially expanded configuration (not shown) when proximal stop 408 and distal stop 409 abut one another, but proximal outer stop 407B and distal stop 410 do not abut. Proximal outer stop 407B may or may abut proximal stop 411. In other words, distal tubular element 404 is not disposed within middle tubular element 403 while middle tubular element 403 is partially or fully nested within proximal tubular element 402.

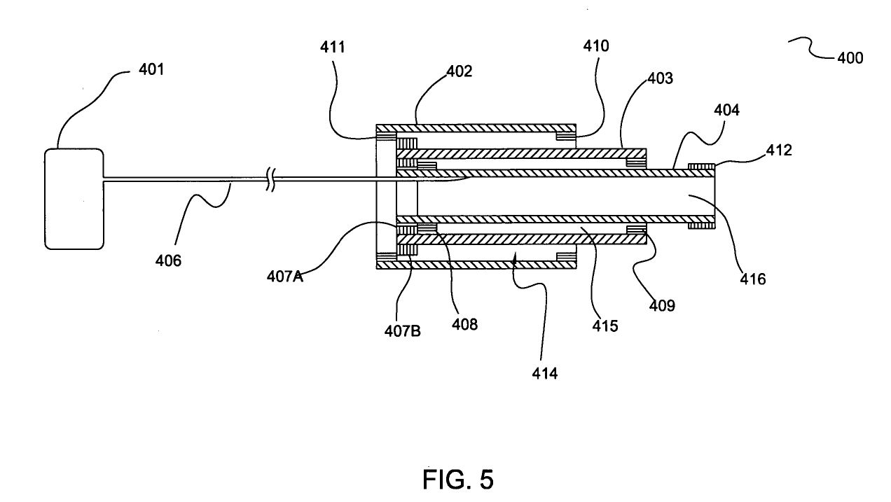

Catheter 400 is in a fully nested or contracted configuration, as shown in FIG. 5, when proximal outer stop 407B and proximal stop 411 abut one another and proximal stop 408 and proximal inner stop 407A abut one another. In other words, distal tubular element 404 is fully nested within middle tubular element 403 and middle tubular element 403 is fully nested within proximal tubular element 402.

The operation and use of catheter 400 as either a deployment or retrieval catheter is very similar to that of catheter 100, described above. When determining the effective over-the-wire length of catheter 100 after insertion, the clinician can choose to extend catheter 400 to any of the lengths available: fully extended or partially extended. Also, for rapid exchange, the clinician may choose to fully retract catheter 400 by drawing positioning wire 406 proximally until catheter 400 is in the fully nested position, or only partially, until one of the partially extended positions is achieved.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents. All patents and publications discussed herein are incorporated in their entirety by reference thereto.

1. A sheath catheter for deploying and/or retrieving a medical device comprising:
a positioning wire having a proximal end and a distal end;
a proximal tube slidably positioned over said positioning wire;
a first lumen extending through said proximal tube, said proximal tube having a first inner diameter;
a distal tube having an outer diameter which is smaller than said first inner diameter of said proximal tube, wherein said positioning wire is fixedly attached to a proximal end of said distal tube, and said distal tube is slidably positionable within said first lumen, and at least a proximal end of said distal tube is unremovably disposed within said first lumen; and
a second lumen extending through said distal tube, wherein a proximal end of said second lumen is open to said first lumen and a distal end of said second lumen disposed at a distal tip of said catheter is configured to allow a medical device to pass therethrough, thereby forming a continuous single lumen extending from a proximal end of said catheter to said distal tip.

2. The sheath catheter according to claim 1, wherein said positioning wire extends into said second lumen and is fixedly attached to an inner wall of said distal tube.

3. The sheath catheter according to claim 1, wherein said proximal tube is made from a polymeric material.

4. The sheath catheter according to claim 3, further including a reinforcing layer.

5. The sheath catheter according to claim 1, wherein said distal tube is made from a polymeric material.

6. The sheath catheter according to claim 5, further including a reinforcing layer.

7. The sheath catheter according to claim 1, wherein said positioning wire is a solid wire.

8. The sheath catheter according to claim 1, wherein a handle is attached to the proximal end of said positioning wire.

9. The sheath catheter according to claim 1, further comprising
a first proximal stop fixedly attached to an interior surface of said proximal tube on a proximal end thereof;
a distal stop fixedly attached to the interior surface of said proximal tube on a distal end thereof; and
a second proximal stop fixedly attached to an exterior surface of said distal tube on a proximal end thereof, wherein said catheter is in a fully extended configuration when the second proximal stop of the distal tube abuts the distal stop of the proximal tube, and said catheter is in a fully contracted configuration when the second proximal stop of the distal tube abuts the first proximal stop of the proximal tube.

10. A sheath catheter for deploying and/or retrieving a medical device comprising:
a positioning wire;
a proximal tube slidably positioned over said positioning wire;
a first lumen extending through said proximal tube, said proximal tube having a first inner diameter;
a middle tube having an outer diameter which is smaller than said first inner diameter of said proximal tube, wherein said middle tube is positioned over said positioning wire distal to said proximal tube, said middle tube is slidably positionable within said first lumen, and at least a proximal end of said middle tube is unremovably disposed within said first lumen;
a second lumen extending through said middle tube, said middle tube having a second inner diameter;
a distal tube, wherein said positioning wire is fixedly attached to a proximal end of said distal tube, said distal tube is slidably positionable within said second lumen, and at least a proximal end of said distal tube is unremovably disposed within said second lumen; and
a third lumen extending through said distal tube, wherein a proximal end of said second lumen is open to said first lumen, a proximal end of said third lumen is open to said second lumen, and a distal end of said third lumen disposed at a distal tip of said catheter is configured to allow a medical device to pass therethrough, thereby forming a continuous single lumen extending from a proximal end of said catheter to said distal tip.

11. The sheath catheter according to claim 10, wherein said positioning wire extends into said third lumen and is fixedly attached to an inner wall of said distal tube.

12. The sheath catheter according to claim 10, further comprising
a first proximal stop fixedly attached to an interior surface of said proximal tube on a proximal end thereof;
a first distal stop fixedly attached to the interior surface of said proximal tube on a distal end thereof;
a second proximal stop fixedly attached to an exterior surface of said middle tube on a proximal end thereof;
a third proximal stop fixedly attached to an interior surface of said middle tube on a proximal end thereof;
a second distal stop fixedly attached to the interior surface of said middle tube on a distal end thereof; and
a fourth proximal stop fixedly attached to an exterior surface of said distal tube on a proximal end thereof, wherein said catheter is in a fully extended configuration when said first distal stop abuts the second proximal stop and the second distal stop abuts the fourth proximal stop.

13. A method for deploying a collapsible medical device within a patient comprising:
expanding a telescoping catheter having a continuous lumen therein to a expanded configuration;
positioning a medical device within said lumen of said telescoping catheter, thereby creating a catheter assembly;
positioning said device within said lumen at or near a distal tip of said telescoping catheter;
inserting said catheter assembly into the patient;
advancing said catheter assembly to a predetermined location within the patient;
contracting said telescoping catheter into a contracted configuration; and
removing said telescoping catheter from the patient.

14. The method according to claim 13, wherein said medical device is diagnostic.

15. The method according to claim 13, wherein said medical device is therapeutic.

16. The method according to claim 13, wherein said medical device is protective.

17. The method according to claim 13, wherein said medical device is collapsible, and inserting said medical device into said lumen causes the medical device to collapse.

18. The method according to claim 13, wherein contracting said telescoping catheter allows the collapsible medical device to expand within the patient.

19. A method for deploying a single-lumen telescoping sheath catheter within a patient comprising:
inserting a proximal end of a guidewire into the lumen; and
pushing a positioning wire fixedly attached to a distal telescoping tube of said telescoping catheter distally, thereby extending the telescoping catheter over said guidewire.

20. A method for removing an indwelling telescoping catheter from a guidewire comprising:
drawing a positioning wire fixedly attached to said indwelling catheter proximally, thereby causing said indwelling catheter to contract to a shorter effective over-the-wire length; and
sliding said catheter proximally over said guidewire until said catheter is removed from said guidewire.
